(19) United States
(12) Patent Application Publication
(10) Pub. No.: US 2005/0131307 A1
(43) Pub. Date: Jun. 16, 2005

(54) Compact oscillometric blood pressure simulator

(76) Inventors: Karl A. Ruiter, South Pasadena, CA (US); William Wallace Ruiter, South Pasadena, CA (US)

Correspondence Address: MICHAEL BLAINE BROOKS, A PROFESSIONAL CORPORATION, 5010 NO. PARKWAY CALABASAS, SUITE 104, CALABASAS, CA, 91302-3913, US

(21) Appl. No.: 11/002,496

(22) Filed: Dec. 1, 2004

Related U.S. Application Data
(60) Provisional application No. 60/529,289, filed on Dec. 15, 2003.

(51) Int. Cl. A61B 5/02; G09B 23/28; A61B 5/00; B65D 81/00

(52) U.S. Cl. 600/485; 434/268; 434/272

(57) ABSTRACT

A system for generating pressure pulses to calibrate a blood pressure (BP) monitor or other pressure sensing device is disclosed. The oscillometric blood pressure (BP) simulator of the preferred embodiment comprises an elastomeric bladder pneumatically coupled to the BP monitor, and an actuator adapted to reversibly compress the elastomeric bladder and induce one or more pressure pulses of predetermined magnitude, duration, and frequency in the BP monitor. The elastomeric bladder generally comprises an elastomeric tube such as medical grade hose or tubing that is compressed by a piston and bearing driven by a linear actuator, preferably a stepper motor and cam. The simulator further includes a plurality of serially coupled H-bridges for energizing the stepper motor windings with bipolar square pulses.

### CROSS-REFERENCE TO RELATED APPLICATION

This application claims the benefit of U.S. Provisional Patent Application Ser. No. 60/529,289 filed Dec. 15, 2003, entitled “COMPACT OSCILLOMETRIC BLOOD PRESSURE SIMULATOR,” which is hereby incorporated by reference herein for all purposes.

### TECHNICAL FIELD

The invention generally relates to a system for generating pulses to simulate the pressure waves transmitted by a blood pressure cuff. In particular, the invention relates to a system for calibrating a blood pressure monitor using a bi-directional actuator and elastomeric coupling to generate user-defined pulses.

### BACKGROUND

Oscillometry refers to the measurement of a patient's blood pressure, typically characterized by the systolic and diastolic blood pressure measurements. Oscillometry is used throughout the medical industry at every stage of patient care to assess cardiovascular function as well as numerous other medical conditions that affect peoples' blood pressure. The most prevalent oscillometers include a blood pressure monitor for pressurizing and controllably deflating a cuff while simultaneously listening for the presence or absence of the pressure pulses caused by the patient's heart beat. Due to the prevalence of blood pressure monitors and the significance of their role in the health care system, there is a need for blood pressure simulators to test and calibrate the blood pressure monitors. Unfortunately, the blood pressure simulators in the prior art suffer from a number of disadvantages. For example, most simulators are unduly large which prevents them from being conveniently carried by a technician to a hospital or within a hospital where the monitor to be tested in located. Some prior art simulators are also able to generate fixed amplitude pressure pulses and therefore lack the flexibility in settings needed to simulate variable blood pressure conditions that may hamper the blood pressure monitor's accuracy. There is therefore a need for a compact, and portable blood pressure simulator adapted to emulate any number of cardiovascular conditions.

### SUMMARY

The invention in the preferred embodiment features a system for generating pulses having a user-defined waveform to simulate the pressure waves acquired by a blood pressure cuff and transmitted to a blood pressure (BP) monitor, for example, thereby permitting the blood pressure monitor to be calibrated. The oscillometric blood pressure (BP) simulator of the preferred embodiment comprises an elastomeric bladder pneumatically coupled to the BP monitor, and an actuator adapted to reversibly compress the elastomeric bladder and induce one or more pressure pulses of a predetermined magnitude, duration, and frequency in the BP monitor. The elastomeric bladder generally comprises an elastomeric tube such as medical grade hose or tubing. The hose or tubing preferably has a circular cross section, although other configurations may also be suitable. Hose and tubing are particular well suited as an elastomeric bladder because they are single-component devices that are easily sealed, highly portable, light-weight, and inexpensive. Any of various types of elastomeric materials may be used including rubber, latex, vinyl, polyester, polypropylene, polyethylene, polyvinyl chloride (PVC), silicone, and nylon, for example.

The simulator actuator in the preferred embodiment includes a linear actuator adapted to precisely compress the elastomeric bladder and induce the one or more pressure pulses in the device under test. The simulator may include a cam coupled to the actuator for driving a piston that directly contacts and compresses the elastomeric bladder. The actuator may be a stepper motor, rotary actuator, linear actuator, solenoid, piezoelectric actuator, or speaker coil actuator, for example.

The simulator of the preferred embodiment further comprises an actuator control mechanism adapted to drive a stepper motor or comparable actuator. Where the stepper motor includes a plurality of windings, the actuator control mechanism may employ a plurality of H-bridges, each of the plurality of H-bridges adapted to drive one of the plurality of stepper motor windings with a bipolar square pulse. When configured in series, the plurality of H-bridges are able to drop substantially all the voltage from commercially available power supplies without the need for a high-current resistor to dissipate the excess power. The actuator control mechanism, in cooperation with the plurality of H-bridges, may be adapted to drive the stepper motor with a sustaining pulse for maintaining the orientation of the cam in a distended position, thereby holding a peak pulse pressure for a finite period of time. The sustaining pulses are adapted to energize the stepper motor windings sufficient to resist the force exerted by the elastomeric bladder, thereby preventing the cam from inadvertently reversing direction when the driving pulses are discontinued. A sustaining pulse generally has a duty cycle less than half the duty cycle of the one or more bi-polar waveforms used to drive the stepper motor in the forward and reverse directions.

In some embodiments, the simulator further includes a microprocessor and a pressure feedback mechanism adapted to control the actuator and induce one or more pressure pulses as the blood pressure monitor deflates the cuff in a step-wise manner. The microprocessor is adapted to induce the one or more pulses in accordance with an oscillometric envelope between a simulated systolic pressure and a simulated diastolic pressure provided by the user.

In a second embodiment, the oscillometric BP simulator includes an actuator, a cam fixedly attached to the actuator, and a piston adapted to induce one or more pressure pulses in the BP monitor. The piston engages the cam via a bearing adapted to operatively couple the piston to the cam with minimal friction and therefore minimal wear at the region of contact. The bearing is preferably fixedly attached to the piston, although it may also be integrally formed with the cam. The bearing preferably includes a ball bearing assembly or a needle bearing assembly, for example.

### BRIEF DESCRIPTION OF THE DRAWINGS

The present invention is illustrated by way of example and not limitation in the figures of the accompanying drawings, and in which:

FIG. 1 is blood pressure simulator, in accordance with the preferred embodiment of the present invention;

FIG. 2 is an exemplary pressure pulse waveform to which the blood pressure monitor is subjected by the simulator, in accordance with the preferred embodiment of the present invention;

FIG. 3 is a graphical depiction of cuff pressure verses time as measured by the blood pressure monitor during an oscillometric measurement cycle, in accordance with the preferred embodiment of the present invention;

FIG. 4 is a motor drive circuit used with the blood pressure simulator, in accordance with the preferred embodiment of the present invention;

FIGS. 5A and 5B are exemplary bi-polar waves used to the excite the first and second windings of the stepper motor, respectively, to drive the cam in the forward direction, in accordance with the preferred embodiment of the present invention; and

FIGS. 6A and 6B are exemplary sustaining pulses that drive the first and second windings of the stepper motor, respectively, to hold the cam in a particular position, in accordance with the preferred embodiment of the present invention.

### DETAILED DESCRIPTION OF THE PREFERRED EMBODIMENT

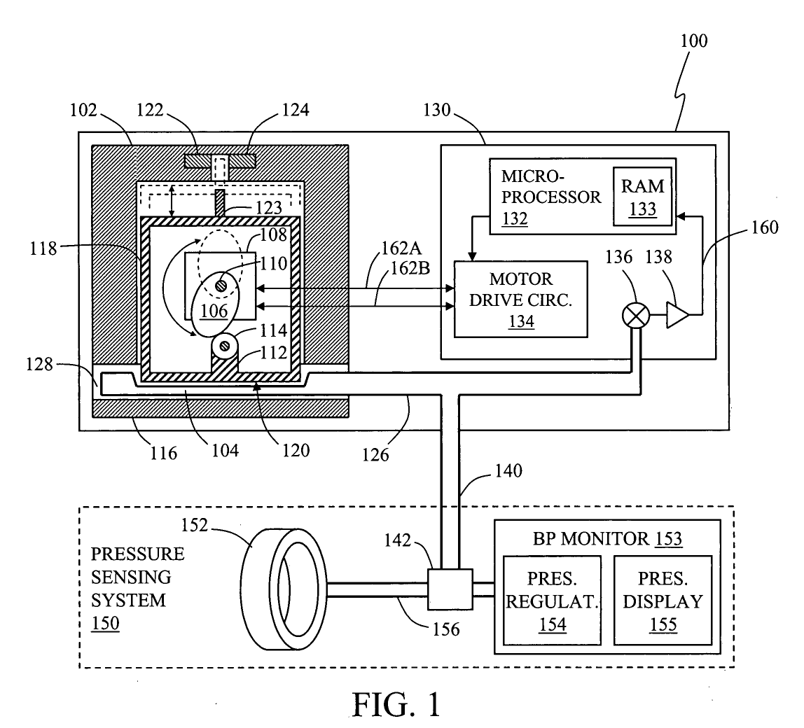

Illustrated in FIG. 1 is the novel pressure simulator adapted to evaluate diagnostic devices ordinarily used to measure pressure or pressure differentials. The preferred embodiment presented herein is an oscillometric blood pressure simulator 100 for testing and calibrating pressure sensing systems including non-invasive blood pressure units 150 used in medical application, for example. The pressure simulator 100 is preferably adapted to generate a pressure waveform including a plurality of pressure pulses to simulate the cardiac rhythm of a patient that would otherwise be acquired by a blood pressure cuff. The pressure simulator 100 is also adapted to simulate signal attenuation attributable to the pressure-dependent impedance mismatch present at the patient-cuff interface. The frequency of the pressure pulses, the diastolic and systolic pressures, and the profile of the pressure pulses may be predetermined by the user to effectively model any of a number of physiological patient conditions for purposes of calibrating blood pressuring monitor equipment regardless of the manufacturer.

An exemplary device under test is represented by the pressure sensing system 150 which includes an inflatable cuff 152 adapted to engage the patient's arm or wrist, for example; and a blood pressure (BP) monitor 153 including a pressure regulator 154 and a pressure display unit 155. The cuff 152 and BP monitor 153 are pneumatically coupled via an elastic hose 156 and T-connector 142. The pressure regulator 154 is adapted to pressurize the cuff 152 above a patient's systolic pressure, slowly dissipate the pressure, and determine the patient's systolic and diastolic pressures which are registered on the pressure display 155.

The pressure simulator 100 preferably includes a bi-directional actuator operatively coupled to the pressure sensing system 150, an actuator control mechanism, and a pressure feedback mechanism. The actuator control mechanism subjects the BP monitor to a plurality of pulses having a determined frequency, magnitude, and profile by controlling the bi-directional actuator under the guidance of the feedback mechanism. In the preferred embodiment of the blood pressure simulator 100, the bi-directional actuator is incorporated into an actuator housing 102 while the actuator control mechanism and a pressure feedback mechanism are incorporated into a simulator control housing 130. Both the actuator housing 102 and control housing 130 are pneumatically coupled to the cuff 152 and BP monitor 153 by means of a pneumatic conduit 126 and extension hose 140. The collective pressure induced by the pressure regulator 154 and the pressure pulses induced by the bi-directional actuator are therefore sensed by the BP monitor 153 as well as the pressure feedback mechanism.

The bi-directional actuator in the preferred embodiment comprises a cam 106 driven by a stepper motor 108. The stepper motor 108 is fixedly attached to the actuator housing 102 and adapted to rotate the motor shaft 110 at least 180 degrees and as much as a full 360 degrees in a clock-wise (CW) or counter clockwise (CCW) manner under the direction of the motor drive control circuit 134. The cam 106 is fixedly attached to the motor shaft 110 and drives a cam follower 112 via a bearing 114 including a ball bearing assembly or needle bearing assembly, for example. The cam follower 112 is fixedly attached to a slider 118 which is concealed within the actuator housing 102 where it is constrained to move solely in the vertical direction. In the preferred embodiment, the displacement of the follower 112 drives the slider 118 up and down within the housing 102, thereby adapting the slider to serve as a piston 120 for exerting a force against the pneumatic chamber 104.

In the preferred embodiment, the pneumatic chamber 104 is formed of an elastomeric material, e.g., rubber or latex hose, that is inserted into a cavity 128 where it is captured between the upper section of the actuator housing 102 and a backing plate 116. The pneumatic chamber 104 in some embodiments is an section of the same hose used for the pneumatic conduit 126. When the cam 106 is turned, the piston 120 compresses the chamber 104 and the increased pressure observed by the pressure monitor 153 as if transmitted from the cuff 152. After the peak pulse pressure is reached, the direction of the cam 106 is reversed and the pressure in the cuff 152 and pneumatic hose 156 restored to the test level controlled by the BP monitor 153. The pressure induced in the cuff 152 is concurrently measured by the pressure feedback mechanism, i.e., the pressure sensor 136, and the sensed pressure signal 160 transmitted to the actuator control mechanism in real-time via the amplifier 138.

In the preferred embodiment, the cam 106 is a linear cam adapted to displace the follower 112 linearly in proportion to the angular displacement of the cam 106. One skilled in the art will appreciate that a non-linear cam 106 may also be employed in alternative embodiments provided a bi-directional actuator is adapted to rotate the cam CW and CCW. In addition to the bi-direction rotary actuator of the preferred embodiment, various other actuators may be employed including linear actuators, solenoids, and piezoelectric devices, for example.

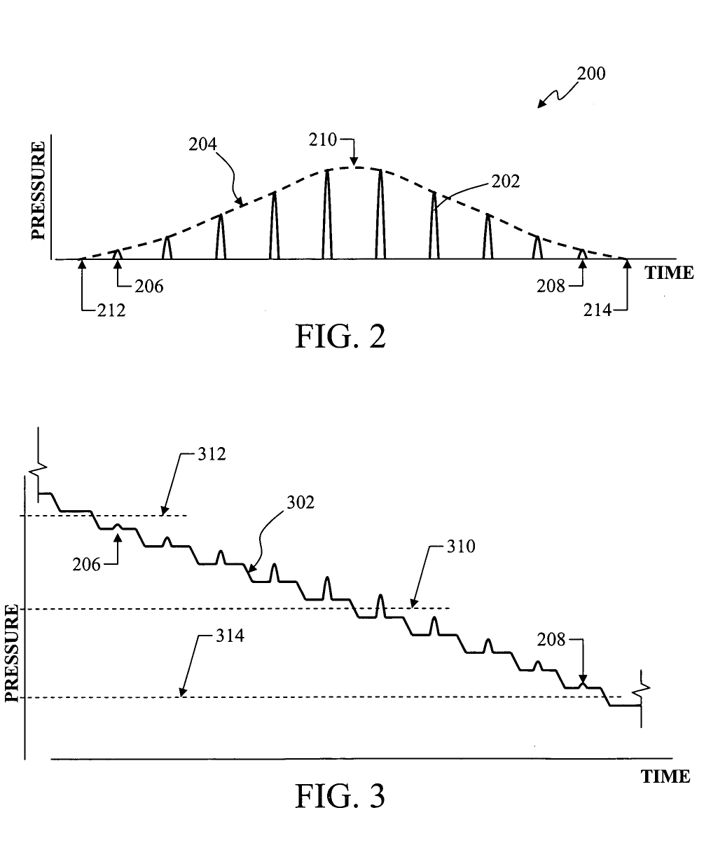

The actuator control mechanism in the preferred embodiment comprises a microprocessor 132 and a motor drive circuit 134. The microprocessor 132 monitors the pneumatic instantaneous pressure reading 160, calculates the size and shape of one or more pressure pulses, and induces the appropriate pulses by exciting the stepper motor 108 via the drive circuit 134. In the preferred embodiment, the size and shape of the induced pressure changes conform to a predetermined pressure waveform retained in local memory, preferably random access memory (RAM) 133. Illustrated in FIG. 2 is an exemplary pressure pulse waveform 200. The waveform 200 includes a plurality of pressure pulses 202 modulated by an oscillometric envelope 204. The oscillometric envelope 204, in turn, is characterized by a mean pressure point 210 and is bounded on the left and right sides by points coinciding with a simulated systolic pressure and a simulated diastolic pressure, respectively. The first pressure pulse 206 of the waveform 200 is introduced at or below the simulated systolic pressure point 212, while the last pulse 208 is introduce prior to or at the simulated diastolic pressure point 214. The train of pressure pulses 202 are generally characterized by a nominal pulse frequency of approximately one Hertz, although one skilled in the art will readily appreciate that the device under test may also be subjected to various other periodic and non-periodic pulse waveforms. The simulated mean pressure point 210 coincides with the point at which a patient's blood pressure optimally matches the cuff pressure and the maximum pulse energy transmitted through the patient-cuff interface. The lower pulse magnitudes on either side of the mean 210 simulate sonic attenuation due to pressure mismatch at the patient-cuff interface that would be observed if an actual patient were being tested.

Illustrated in FIG. 3 is a graphical depiction of cuff pressure verses time as measured by the blood pressure unit 150 during an oscillometric measurement cycle. The cuff 152 is initially inflated above the simulated systolic pressure 312 and the pressure released from the cuff 152 in a step-wise manner. When the simulator 100 detects that the cuff pressure 302 has dropped to the simulated systolic pressure 312, the microprocessor 132 causes the drive circuit 134 to turn the cam 106 in a CCW direction wherein the piston 120 deforms the pneumatic chamber 104, thus introducing the first pressure pulse 206. The direction of the cam 106 is reversed after the cuff pressure 302 has been increased by the magnitude of the first pressure pulse 206 given in FIG. 2. As the cuff pressure continues to decrease under the control of the pressure regulator 154, the magnitude of the pressure pulses injected by the actuator control mechanism increases in accordance with the oscillometric envelope 204 until the ambient cuff pressure equals the simulated mean pressure 310 which coincides with the mean pressure point 210. As the cuff pressure further decreases, the actuator control mechanism further reduces the magnitude of the pressure pulses until the last pulse 208 is injected and the ambient cuff pressure falls below the simulated diastolic pressure 314. The simulated systolic pressure and simulated diastolic pressure may then be compared to the measured pressures determined by the BP monitor 153 for purposes of calibrating the monitor, for example.

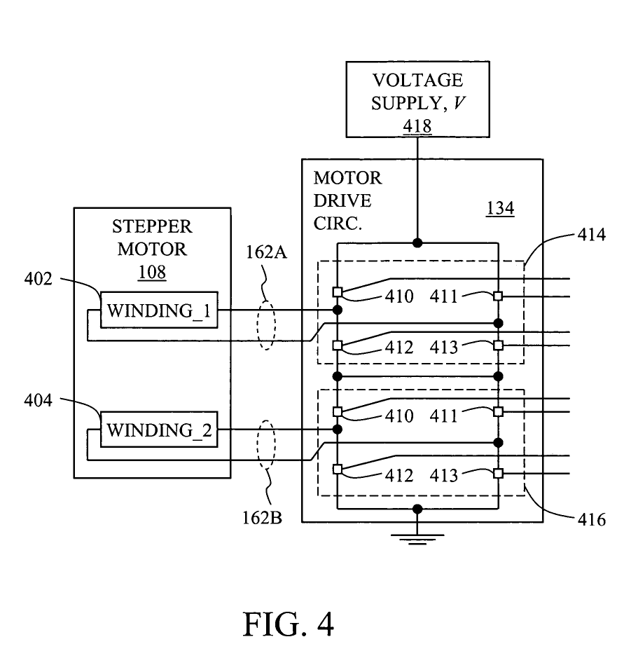

Referring to FIG. 4, the bi-directional actuator 108 in the preferred embodiment is a stepper motor including two sets of winding schematically represented by the first winding 402, i.e., WINDING_{—}1, and a second winding 404, i.e., WINDING_{—}2. The windings 402, 404 may be excited in different sequences and with different polarities to obtain various possible motor speed in both the forward and reverse directions. In the preferred embodiment, the two windings 402, 404 are adapted to receive simultaneous bi-polar square waves from the motor drive circuit 134, the first square wave 162A being 90 degrees out of phase with respect to the second square wave 162B. The number of cycles of the bi-polar square pulses determines the angular displacement of the cam 106 while the polarity of the phase difference between the square waves 162A, 162B determines the direction of rotation.

In the preferred embodiment the motor drive circuit 134 includes two H-bridges for toggling the polarity of the bi-polar square waves used to control the stepper motor 108. The first H-bridge 414 controls or drives the first winding 402 while the second H-bridge 416 controls or drives the second winding 404. In the novel implementation of the preferred embodiment, the H-bridges 414, 416 are coupled in series instead of parallel. Each of the H-bridges includes four switches 410-413 made to open or close under the direction of the motor drive circuit 134. In steady state, the switches are toggled every half cycle, T/2, where the full period, T, is determined by the temporal resolution of the selected stepper motor 108.

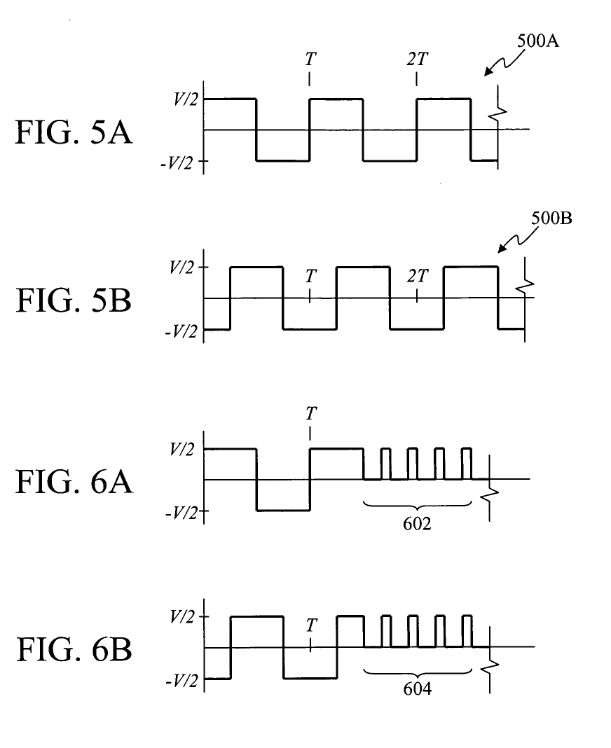

To drive the cam 106 in a CCW direction, for example, switches 410, 413 of the first H-bridge 414 are closed while switches 411, 412 of the first H-bridge 414 are opened. Every half cycle, T/2, the switches are 410-413 are toggled, thus giving rise to the bi-polar square pulse 500A illustrated in FIG. 5A. Concurrently, the switches 410, 413 of the second H-bridge 416 are opened while switches 411, 412 of the second H-bridge 416 are closed. The switches 410-413 of the second H-bridge 416 are toggled after a quarter cycle, T/4, and toggled every half cycle thereafter, thus giving rise to the bi-polar square wave 500B illustrated in FIG. 5B. To drive the cam 106 in the CW direction, relative phase of the bi-polar drive signals is reversed, i.e., the first wave 500A inputted to the first winding 402 is made to lag the second wave 500B inputted to the second winding 404 by a ninety degrees. Whether the cam 106 is driven CC or CCW, each of the windings 402, 404 drops one half the system voltage, V, provided by the power supply 418. Coupling the H-bridges 414, 416 in series instead of parallel obviates the need for high-current resistors used in the prior art to drop the voltage difference between the power supply and a single winding.

Referring to FIGS. 6A and 6B, the actuator control mechanism of the preferred embodiment is also adapted to produce one or more sustaining pulses used to hold the cam in a desired position. In particular, sustaining pulses are employed to maintain the cam in position while resisting the torque created by the piston 120 when it is engaged against the pneumatic chamber 104. As such, the cam 106 may be locked in position to extend the width of one or more pressure pulses 202. In the preferred embodiment, the sustaining pulses 602, 604 are represented by intermittent square pulses that are enabled when the cam has reached the position to be maintained. In the preferred embodiment, the sustaining pulses 602, 604 are square pulses having the same amplitude as their respective bi-polar square pulses immediately prior to the initiation of the sustaining pulses. The sustaining pulses are preferably uni-polar, have a pulse width of approximately T/12, and have a repetition period of approximately T/6. After the sustaining pulses, the cam 106 may be driven in the reverse direction to its home position, for example.

The implementation of the novel sustaining pulses presented herein allows the pressure simulator 100 extend its pulse duration while appreciably reducing its power consumption. As such, the voltage source 410 may employ disposable batteries, thus increasing its portability while reducing its cost.

Referring to FIG. 1 again, the pressure simulator 100 in some embodiments further includes a photo interrupt detector that insures the cam 106 returns to the same position—termed the home-position—at the onset of each pressure pulse. The sensor in the preferred embodiment includes a light source, e.g., a light emitting diode (LED) 122, a light detector, e.g., a photovoltaic cell 124, and an opaque blade 123 fixedly attached to the slider 118. When the cam 106 is away from it home position and engaged against the pneumatic chamber 104, for example, the position of the blade 123 permits the cell 124 to receive the light emitted by the LED 122. When the slider 118 is biased towards the upper side of the housing 102, however, the opaque blade 123 interrupts the light beam between the LED 122 and cell 124, thus confirming that the cam 106 is located in the home position. Once the home position is confirmed, the cam 106 position is initialized before it is advanced to one of a plurality of possible staging positions where it will await the initiation of the next pressure pulse. In the preferred embodiment, the slider 118 may be staged: (a) in immediate proximity to the pneumatic chamber 104 or (b) in a position biased slightly against the pneumatic chamber. In the latter case, the piston 120 induces minor deformation of the pneumatic chamber 104 which enables the simulator 100 to induce the peak pulse pressure more rapidly compared to the alternate staging position or the home position. The simulator 100 may be designed such that the angular offset between the home position and either of the two staging positions is given by a predetermined number of stepper motor increments to which the cam 106 may be automatically advanced preceding the initiation of a pressure pulse.

Although the description above contains many specifications, these should not be construed as limiting the scope of the invention but as merely providing illustrations of some of the presently preferred embodiments of this invention.

Therefore, the invention has been disclosed by way of example and not limitation, and reference should be made to the following claims to determine the scope of the present invention.

1. An oscillometric blood pressure (BP) simulator adapted to test a BP monitor, the simulator comprising:
an elastomeric bladder pneumatically coupled to the BP monitor; and
an actuator adapted to reversibly compress the elastomeric bladder;
wherein the compressed elastomeric bladder is adapted to induce one or more pressure pulses in the BP monitor.

2. The simulator in claim 1, wherein the elastomeric bladder consists essentially of an elastomeric tube.

3. The simulator in claim 2, wherein the elastomeric tube is selected from the group comprising: rubber tubing, a latex tubing, a vinyl tubing, polyester tubing, polypropylene tubing, polyethylene tubing, polyvinyl chloride tubing, silicone tubing, and nylon tubing.

4. The simulator in claim 1, wherein the elastomeric bladder comprises a substantially circular cross section in a region contacted by the actuator.

5. The simulator in claim 1, wherein the actuator is a linear actuator.

6. The simulator in claim 1, wherein the simulator further comprises:
a cam operatively coupled to the actuator; and
a piston operatively coupled to the cam, wherein the piston is adapted to compress the elastomeric bladder.

7. The simulator in claim 1, wherein the actuator is selected from the group consisting of: rotary actuators, linear actuators, solenoids, piezoelectric actuators, and speaker coil actuators.

8. The simulator in claim 1, wherein the actuator comprises a stepper motor.

9. The simulator in claim 8, wherein the simulator further comprises an actuator control mechanism adapted to drive the stepper motor.

10. The simulator in claim 9, wherein the stepper motor comprises a plurality of windings, and wherein the actuator control mechanism comprises a plurality of H-bridges, each of the plurality of H-bridges adapted to drive one of the plurality of stepper motor windings.

11. The simulator in claim 10, wherein the plurality of H-bridges are serially coupled.

12. The simulator in claim 10, wherein the simulator is adapted to drive the stepper motor with a sustaining pulse for maintaining an orientation of a cam.

13. The simulator in claim 1, wherein the simulator further comprises a microprocessor adapted to control the actuator to induce one or more pressure pulses between a simulated systolic pressure and a simulated diastolic pressure.

14. The simulator in claim 13, wherein the one or more pressure pulses conform to an oscillometric envelope.

15. The simulator in claim 1, wherein the simulator further comprises a pressure sensor adapted to measure BP monitor pressure.

16. An oscillometric blood pressure (BP) simulator adapted to test a BP monitor, the simulator comprising:
an actuator;
a cam fixedly attached to the actuator; and
a piston adapted to induce one or more pressure pulses in the BP monitor, the piston operatively coupled to the cam via a bearing.

17. The simulator in claim 16, wherein the bearing is fixedly attached to the piston.

18. The simulator in claim 16, wherein the bearing is selected from a group consisting of:
a ball bearing assembly and a needle bearing assembly.

19. An oscillometric blood pressure (BP) simulator adapted to test a BP monitor, the simulator comprising:
a stepper motor adapted to induce one or more pressure pulses in the BP monitor via a cam fixedly attached to the actuator; and
an actuator control mechanism adapted to drive the stepper motor in a forward direction with one or more bi-polar waveforms, and adapted to maintain an orientation of the cam with one or more sustaining pulses.

20. The simulator in claim 19, wherein the one or more sustaining pulses have a duty cycle less than half a duty cycle of the one or more bi-polar waveforms.
